(19) United States
(12) Patent Application Publication
(10) Pub. No.: US 2024/0180466 A1
(43) Pub. Date: Jun. 6, 2024

(54) Three-Dimensional Animated Personality Assessment

(71) Applicant: Paradox, Inc., Scottsdale, AZ (US)

(72) Inventors: Justin Marshall, Edmond, OK (US); Scott Tremper, Costa Mesa, CA (US); Dave Volpe, Middle River, MD (US); Heather Myers, Mountain View, CA (US); Mark Parkinson, Glastonbury (GB); Rachel Stewart Johnson, Poway, CA (US); Daniel Sines, Middle River, MD (US); Joshua Spears, Baltimore, MD (US)

(73) Assignee: Paradox, Inc., Scottsdale, AZ (US)

(21) Appl. No.: 18/523,357

(22) Filed: Nov. 29, 2023

Related U.S. Application Data
(60) Provisional application No. 63/385,900, filed on Dec. 2, 2022.

(51) Int. Cl. A61B 5/16; G16H 10/20

(52) U.S. Cl. CPC A61B 5/167; G16H 10/20

(57) ABSTRACT

A computing system receives a plurality of customized three-dimensional image-caption pairs generated by an administrator. Each customized three-dimensional image-caption pair may be assigned a plurality of traits to each of the plurality of customized three-dimensional image-caption pairs. For each customized three-dimensional image-caption pair, the computing system scores each trait of the plurality of traits. The computing system deploys a personality assessment. The personality assessment includes the plurality of customized three-dimensional image-caption pairs. For each customized three-dimensional image-caption pair, the computing system receives response data from a test-taker. The response data includes an indication of whether the test-taker relates to the customized three-dimensional image-caption pair. Based on the response data, the computing system categorizes the test-taker into a personality dimension or personality type.

### CROSS-REFERENCE TO RELATED APPLICATIONS

This application claims priority to U.S. Provisional Application Ser. No. 63/385,900, filed Dec. 2, 2022, which is hereby incorporated by reference in its entirety.

### FIELD OF THE DISCLOSURE

Embodiments disclosed herein generally related to a personality assessment system and, more specifically, to a personality assessment system with three-dimensional animated images.

### BACKGROUND

Personality assessments are used across industries to not only assess whether an individual is a current fit for a particular organization or entity, but also whether the individual is a fit for a particular role within an organization or entity. By leveraging personality assessments organizations can determine how to best utilize its employees, such that the organization's employees are set up to succeed within the organization.

### SUMMARY

In some embodiments, a method is disclosed herein. A computing system receives a plurality of customized three-dimensional image-caption pairs generated by an administrator. Each three-dimensional image-caption pair may be associated with a plurality of traits assigned thereto. For each customized three-dimensional image-caption pair, the computing system scores each trait of the plurality of traits. The computing system deploys a personality assessment. The personality assessment includes the plurality of customized three-dimensional image-caption pairs. For each customized three-dimensional image-caption pair, the computing system receives response data from a test-taker. The response data includes an indication of whether the test-taker relates to the customized three-dimensional image-caption pair. Based on the response data, the computing system categorizes the test-taker into a personality dimension or personality type.

In some embodiments, a non-transitory computer readable medium is disclosed herein. The non-transitory computer readable medium includes one or more sequences of instructions, which, when executed by one or more processors, causes a computing system to perform operations. The operations include receiving, by the computing system, a plurality of customized three-dimensional image-caption pairs generated by an administrator. Each three-dimensional image-caption pair may be associated with a plurality of traits assigned thereto. The operations further include, for each customized three-dimensional image-caption pair, scoring, by the computing system, each trait of the plurality of traits. The operations further include deploying, by the computing system, a personality assessment. The personality assessment includes the plurality of customized three-dimensional image-caption pairs. The operations further include, for each customized three-dimensional image-caption pair, receiving, by the computing system, response data from a test-taker. The response data includes an indication of whether the test-taker relates to the customized three-dimensional image-caption pair. The operations further include, based on the response data, categorizing, by the computing system, the test-taker into a personality dimension or personality type.

In some embodiments, a system is disclosed herein. The system includes a processor and a memory. The memory has programming instructions stored thereon, which, when executed by the processor, causes the system to perform operations. The operations include receiving a plurality of customized three-dimensional image-caption pairs generated by an administrator. Each three-dimensional image-caption pair may be associated with a plurality of traits assigned thereto. The operations further include, for each customized three-dimensional image-caption pair, scoring each trait of the plurality of traits. The operations further include deploying a personality assessment. The personality assessment includes the plurality of customized three-dimensional image-caption pairs. The operations further include, for each customized three-dimensional image-caption pair, receiving response data from a test-taker. The response data includes an indication of whether the test-taker relates to the customized three-dimensional image-caption pair. The operations further include, based on the response data, categorizing the test-taker into a personality dimension or personality type.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1 is a block diagram illustrating a computing environment, according to one exemplary embodiment.

FIG. 2 is a block diagram illustrating back-end computing system of FIG. 1, according to example embodiments.

FIG. 3A illustrates an exemplary customized image-caption pair, according to example embodiments.

FIG. 3B illustrates an exemplary customized image-caption pair, according to example embodiments.

FIG. 3C illustrates an exemplary customized image-caption pair, according to example embodiments.

FIG. 4 is a flow diagram illustrating a method of facilitating a personality assessment for a test taker, according to example embodiments.

FIG. 5A illustrates a system bus computing system architecture, according to example embodiments.

FIG. 5B illustrates a computer system having a chipset architecture, according to example embodiments.

To facilitate understanding, identical reference numerals have been used, where possible, to designate identical elements that are common to the figures. It is contemplated that elements disclosed in one embodiment may be beneficially utilized on other embodiments without specific recitation.

### DETAILED DESCRIPTION

Traditionally, personality assessment systems utilize text-based questions to measure a user's personality. As personality assessment systems have improved, these systems have begun to adopt a visual-based personality assessment that asks test-takers to respond to a series of image. Usually, these personality assessment systems are limited in the types of images they are able to deliver to a user. For example, existing personality assessment systems typically use stock photography taken from external systems, such as, but not limited to gettyimages from Getty Images, Inc. As those skilled in the art appreciate, relying on a third-party system's corpus of images limits the types of scenarios a personality assessment system can provide to individuals. As a result, the final personality assessment may be limited or incomplete.

To address the deficiencies of existing personality assessment systems, one or more techniques provided herein allow administrators to create custom three-dimensional animated images to replace the stock images that are traditionally used. By providing a personality assessment system that allows administrators to create custom 3D animated images, the personality assessment experience can be tailored to allow each image to precisely capture the concept being relayed to the test-taker and thus produces a more accurate and predictive output that can be used to evaluate the test-takers personality and fitness for various roles.

The term “user” or “test-taker” as used herein includes, for example, a person or entity that owns a computing device or wireless device; a person or entity that operates or utilizes a computing device or wireless device; or a person or entity that is otherwise associated with a computing device or wireless device. It is contemplated that the term “user” or “test-taker” is not intended to be limiting and may include various examples beyond those described.

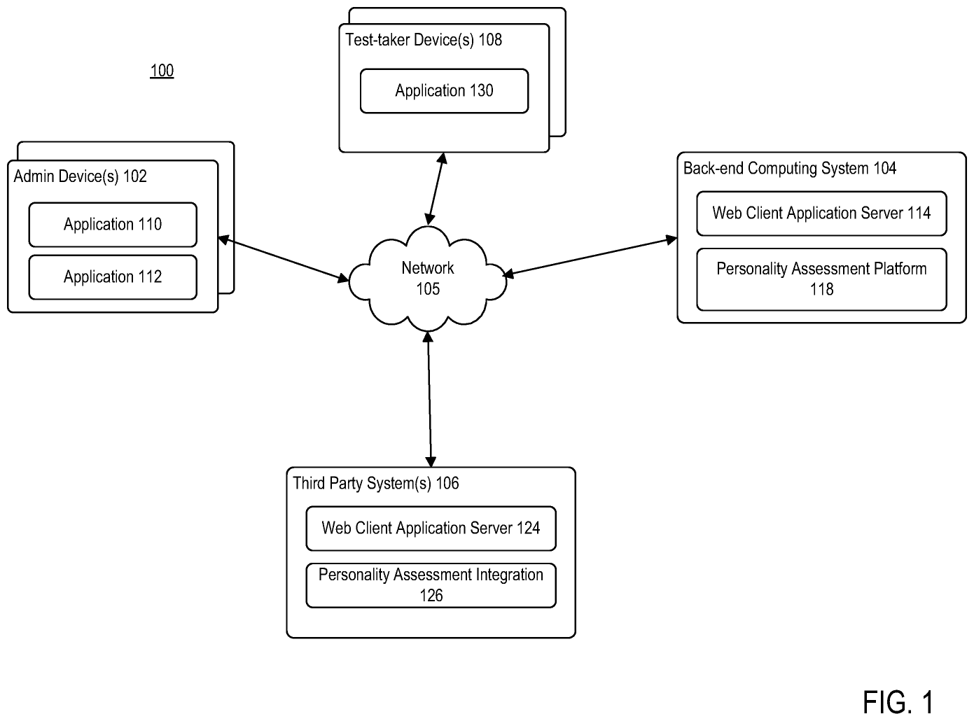

FIG. 1 is a block diagram illustrating computing environment 100, according to one embodiment. Computing environment 100 may include at least one or more administrator (“admin”) devices 102, a back-end computing system 104, a third-party system 106, and one or more test-taker devices 108 communicating via network 105.

Network 105 may be of any suitable type, including individual connections via the Internet, such as cellular or Wi-Fi networks. In some embodiments, network 105 may connect terminals, services, and mobile devices using direct connections, such as radio frequency identification (RFID), near-field communication (NFC), Bluetooth™, low-energy Bluetooth™ (BLE), Wi-Fi™, ZigBee™, ambient backscatter communication (ABC) protocols, USB, WAN, or LAN. Because the information transmitted may be personal or confidential, security concerns may dictate one or more of these types of connection be encrypted or otherwise secured. In some embodiments, however, the information being transmitted may be less personal, and therefore, the network connections may be selected for convenience over security.

Network 105 may include any type of computer networking arrangement used to exchange data. For example, network 105 may be the Internet, a private data network, virtual private network using a public network and/or other suitable connection(s) that enables components in computing environment 100 to send and receive information between the components of computing environment 100.

Admin device 102 may be operated by an administrator. In some embodiments, admin device 102 may be operated by an administrator of an entity associated with back-end computing system 104. For example, admin device 102 may be operated by an administrator of an entity that delivers the personality assessment to test-takers. In some embodiments, admin device 102 may be operated by an administrator of an entity associated with third party system 106. Admin device 102 may be representative of a mobile device, a tablet, a desktop computer, or any computing system having the capabilities described herein.

Admin device 102 may include at least application 110 and application 112. In some embodiments, application 110 may be representative of a graphics software locally installed and executing on admin device 102. In some embodiments, application 110 may be representative of a cloud-based or remote graphics software accessed via application 110. Application 110 may be representative of any graphics software. Exemplary graphics software may include, but is not limited to, Autodesk Maya, Adobe Photoshop, Arnold Render, Substance Painter, ZBrush, Phoenix FD, Vray, and the like. Via application 110, an admin may generate custom images for inclusion in a personality assessment. In some embodiments, the custom images may be three-dimensional animated images. By creating custom images, the assessment experience can be fine-tuned with images that precisely capture the concept being relayed to the test-taker. In this manner, the custom images may produce a more accurate and predictive output that can be used to evaluate the test-takers personality and fitness for various roles.

Application 112 may be representative of an application associated with back-end computing system 104. In some embodiments, application 112 may be a standalone application associated with back-end computing system 104. In some embodiments, application 112 may be representative of a web-browser configured to communicate with back-end computing system 104. In some embodiments, admin device 102 may communicate over network 105 to request a webpage, for example, from web client application server 114 of back-end computing system 104. For example, admin device 102 may be configured to execute application 112 to upload custom images and/or access personality assessments managed by web client application server 114. The content that is displayed to admin device 102 may be transmitted from web client application server 114 to admin device 102, and subsequently processed by application 112 for display through a graphical user interface (GUI) of admin device 102.

Test-taker device 108 may be associated with a test-taker. A “test-taker” may refer to a user who is going to, is, or will take a personality assessment managed by back-end computing system 104. Test-taker device 108 may be representative of a mobile device, a tablet, a desktop computer, or any computing system having the capabilities described herein. Test-taker device 108 may include at least application 130.

In some embodiments, application 130 may be representative of an application associated with back-end computing system 104. In some embodiments, application 130 may be a standalone application associated with back-end computing system 104. In some embodiments, application 130 may be representative of a web-browser configured to communicate with back-end computing system 104. In some embodiments, test-taker device 108 may communicate over network 105 to request a webpage, for example, from web client application server 114 of back-end computing system 104. For example, test-taker device 108 may be configured to execute application 130 to access a personality assessment managed by web client application server 114. The content that is displayed to test-taker device 108 may be transmitted from web client application server 114 to test-taker device 108, and subsequently processed by application 130 for display through a graphical user interface (GUI) of test-taker device 108.

In some embodiments, application 130 may be representative of an application associated with a third-party system 106. For example, rather than interface with back-end computing system 104 directly, test-taker device 108 may interface with functionality of back-end computing system 104 via a third-party integration on a third-party website. In some embodiments, application 130 may be a standalone application associated with third party system 106. In some embodiments, application 130 may be representative of a web-browser configured to communicate with third party system 106. In some embodiments, test-taker device 108 may communicate over network 105 to request a webpage, for example, from web client application server 124 of third-party system 106. For example, test-taker device 108 may be configured to execute application 130 to access a personality assessment through a website associated with third party system 106. The content that is displayed to test-taker device 108 may be transmitted from web client application server 124 and/or web client application server 114 to test-taker device 108, and subsequently processed by application 130 for display through a graphical user interface (GUI) of test-taker device 108.

Back-end computing system 104 may include web client application server 114 and personality assessment platform 118. Personality assessment platform 118 may be configured to create and manage personality assessments for end-users. In some embodiments, personality assessment platform 118 may be accessed by one or more admin devices 102 for creating a personality assessment. In some embodiments, personality assessment platform 118 may be accessed by one or more test-taker devices 108 for taking a personality assessment hosted by personality assessment platform 118.

Third party systems 106 may be associated with one or more third party entities. Each third-party system 106 may include web client application server 124 and personality assessment integration 126. Personality assessment integration 126 may allow third party system 106 to incorporate functionality of personality assessment platform 118 into a website associated with third party system 106 or content presented via application 130 associated with third party system 106. For example, functionality of personality assessment platform 118 may be incorporated into webpages of websites associated with third party system 106 (e.g., job boards, career sites, etc.), web-based platforms, and the like.

In some embodiments, personality assessment integration 126 may take the form of code that may be injected into the web code of a website hosted by web client application server 124. In another example, personality assessment integration 126 may take the form of code that may be injected into the code of application 130 executing on test-taker device 108. Once injected into the web code of the website or code of application 130, a personality assessment supported by back-end computing system 104 may be incorporated into content provided by third party system 106. As such, when a user of test-taker device 108 accesses application 130, application 130 may render information associated with a personality test hosted by back-end computing system 104 within application 130. In some embodiments, when a user of test-taker device 108 requests a website from web client application server 124, application 130 may render a personality test hosted by back-end computing system 104 within the website.

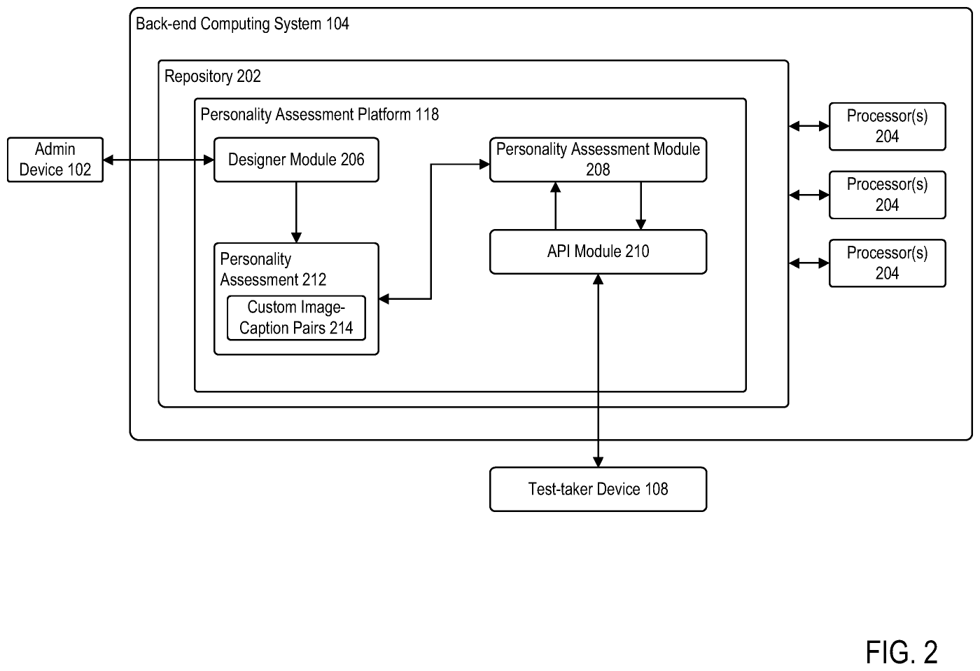

FIG. 2 is a block diagram illustrating back-end computing system 104, according to example embodiments. As shown, back-end computing system 104 includes repository 202 and one or more computer processors 204.

Repository 202 may be representative of any type of storage unit and/or device (e.g., a file system, database, collection of tables, or any other storage mechanism) for storing data. Further, repository 202 may include multiple different storage units and/or devices. The multiple different storage units and/or devices may or may not be of the same type or located at the same physical site. As shown, repository 202 includes at least personality assessment platform 118.

Personality assessment platform 118 may include designer module 206, personality assessment module 208, and application programming interface (API) module 210. Each of designer module 206, personality assessment module 208, and API module 210 may be comprised of one or more software modules. The one or more software modules are collections of code or instructions stored on a media (e.g., memory of back-end computing system 104) that represent a series of machine instructions (e.g., program code) that implements one or more algorithmic steps. Such machine instructions may be the actual computer code the processor of back-end computing system 104 interprets to implement the instructions or, alternatively, may be a higher level of coding of the instructions that are interpreted to obtain the actual computer code. The one or more software modules may also include one or more hardware components. One or more aspects of an example algorithm may be performed by the hardware components (e.g., circuitry) itself, rather than as a result of the instructions.

Designer module 206 may be configured to assist a user in designing or generating a personality assessment. For example, as shown, admin device 102 may access personality assessment platform 118 to create a personality assessment. In some embodiments, admin device 102 may upload a three-dimensional graphic image that was generated by the admin using application 110. In some embodiments, admin device 102 may provide a caption with the three-dimensional graphic image. The caption may describe a trait, sentiment, or idea captured by the image.

In some embodiments, an admin may provide the various traits that are assigned or tagged to the image-caption pair. Each trait corresponds to an overarching personality dimension or personality type. In some embodiments, an admin may assign 5-8 personality traits to the image-caption pair. In some embodiments, an admin may assign more than 8 personality traits the image-caption pair. In some embodiments, an admin may assign less than 5 personality traits to the image-caption pair. In some embodiments, an admin may score each of the assigned personality traits. For example, an admin may rank each personality trait based on an analysis of the image. The personality trait's rank may correspond to a score associated with that personality trait for that image. Exemplary personality traits may include, but are not limited to, compassionate, loyal, open-minded, thrill-seeking, and altruistic.

Once uploaded, one or more admins (e.g., a plurality of admin devices 102) may access the image-caption pair to test the image. For example, various admins may test the image-caption pair to determine whether the image-caption pair accurately conveys the intent of the image-caption pair. In some embodiments, admins may perform such testing using external test-takers. Based on the testing, admin devices 102 may either approve the image-caption pair, adjust the image-caption pair, or discard the image-caption pair.

In those situations where an admin device 102 determines that an adjustment is needed, admin device 102 may adjust one or more of the three-dimensional graphical image, the caption, the ranking of personality traits, or the personality traits themselves.

As output, designer module 206 may generate a personality assessment 212 for deployment. Personality assessment 212 may include a plurality of custom image-caption pairs 214. For example, personality assessment 212 may include a plurality of image-caption pairs 214 that includes a custom three-dimensional graphic image generated by a user of admin device 102.

Personality assessment module 208 may be configured to manage the personality assessments provided by personality assessment platform 118. For example, personality assessment module 208 may be configured to manage personality assessment 212. Managing personality assessment 212 may include personality assessment module 208 deploying personality assessment 212 to test-taker devices 108.

In some embodiments, personality assessment module 208 may deploy personality assessment via API module 210. For example, if test-taker device 108 accesses personality assessment 212 via a website associated with third party system 106, personality assessment 212 may be delivered to test-taker device 108 through the website via one or more APIs managed by API module 210. In this manner, personality assessment module 208 may receive user responses from API module 210.

Personality assessment module 208 may further be configured to generate a personality dimension or personality type of the test-taker based on test-taker feedback to personality assessment 212. For example, each custom image-caption pair 214 is provided to the user via a user interface of test-taker device 108. When presented, custom image-caption pair 214 may prompt the test-taker for feedback. In some embodiments, the test-taker prompt may be “me” or “not me.” In other words, the test-taker for feedback regarding whether the custom image-caption pair 214 is the test-taker (“me”) or not the test-taker (“not me”).

Personality assessment module 208 may collect personality trait data with each test-taker response, i.e., with each “me” or “not me” response to each custom image-caption pair 214. Based on the personality trait data, personality assessment module 208 may categorize the test-taker into a personality dimension or personality type. For example, personality assessment module 208 may apply an algorithm to the test-takers feedback, which may aggregate the personality traits and respective scores for each custom image-caption pair 214 and output a personality dimension or personality type. Exemplary personality dimensions may include, but are not limited to, openness, conscientiousness, extraversion, agreeableness, emotional stability. Exemplary personality types may include, but are not limited to, visionary, planner, action-taker, inventor, mentor, and analyzer.

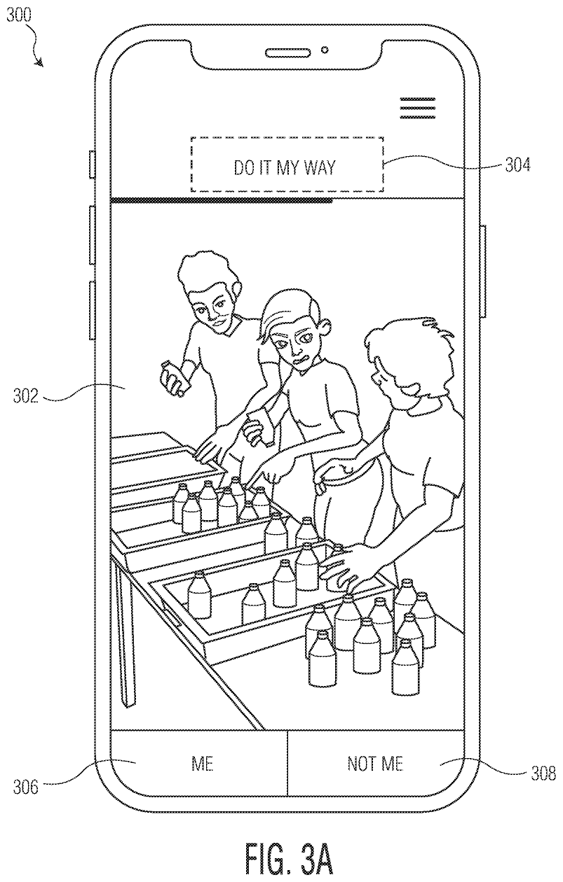

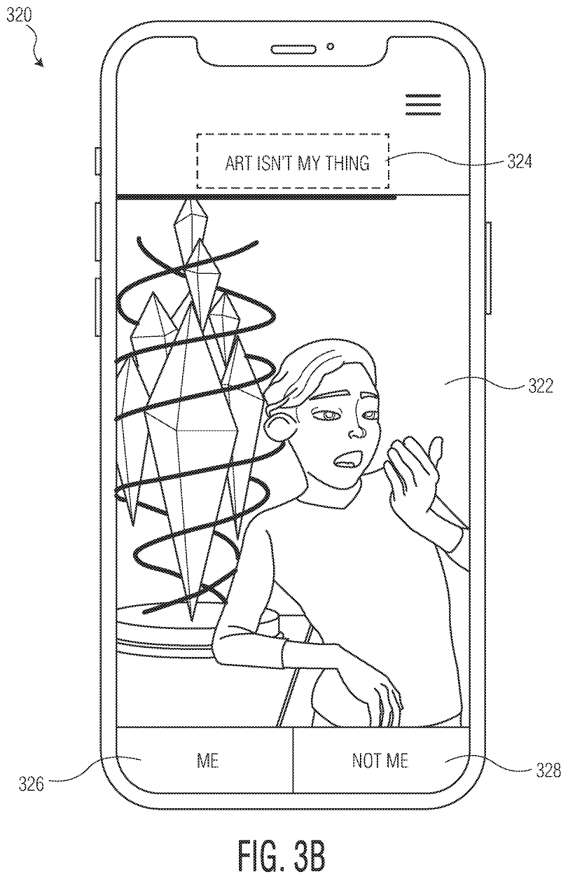

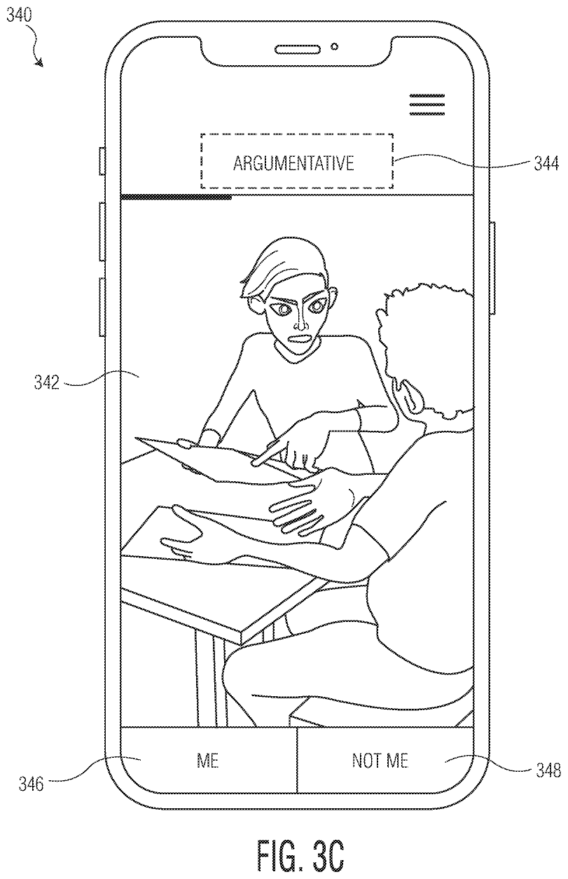

FIG. 3A illustrates an exemplary customized image-caption pair 300, according to example embodiments. Customized image-caption pair 300 may be delivered to test-taker device 108 via application 130 executing thereon. In some embodiments, customized image-caption pair 300 may be delivered to test-taker device 108 via a third-party website.

As illustrated, customized image-caption pair 300 may include a customized three-dimensional image 302 and a caption 304. Customized three-dimensional image 302 may be generated by an admin device 102 using a graphical application 110. In image-caption pair 300, the designer or admin attempted to capture an individual working in a group setting. Here, the message conveyed to the test-taker is whether the sentiment “do it my own way” in a group work setting would be their sentiment to this scenario.

The test-taker can provide feedback via graphical element 306 or graphical element 308. For example, graphical element 306 may correspond to a “me” response. In other words, the test-taker relates to the sentiment illustrated in customized image-caption pair 300. Graphical element 308 may correspond to a “not me” response. In other words, the test-taker does not relate to the sentiment illustrated in customized image-caption pair 300.

FIG. 3B illustrates an exemplary customized image-caption pair 320, according to example embodiments. Customized image-caption pair 320 may be delivered to test-taker device 108 via application 130 executing thereon. In some embodiments, customized image-caption pair 320 may be delivered to test-taker device 108 via a third-party website.

As illustrated, customized image-caption pair 320 may include a customized three-dimensional image 322 and a caption 324. Customized three-dimensional image-caption pair 320 may be generated by an admin device 102 using a graphical application 110. In image-caption pair 300, the designer or admin attempted to capture an individual “looking” at a piece of art. Here, the message conveyed to the test-taker is whether the test-taker relates to the sentiment “art isn't my thing.”

The test-taker can provide feedback via graphical element 326 or graphical element 328. For example, graphical element 326 may correspond to a “me” response. In other words, the test-taker relates to the sentiment illustrated in customized image-caption pair 320. Graphical element 328 may correspond to a “not me” response. In other words, the test-taker does not relate to the sentiment illustrated in customized image-caption pair 320.

FIG. 3C illustrates an exemplary customized image-caption pair 340, according to example embodiments. Customized image-caption pair 340 may be delivered to test-taker device 108 via application 130 executing thereon. In some embodiments, customized image-caption pair 340 may be delivered to test-taker device 108 via a third-party website.

As illustrated, customized image-caption pair 340 may include a customized three-dimensional image 342 and a caption 344. Customized three-dimensional image-caption pair 340 may be generated by an admin device 102 using a graphical application 110. In image-caption pair 340, the designer or admin attempted to capture an individual interacting with a colleague. Here, the message conveyed to the test-taker is whether the test-taker relates to the sentiment “argumentative” when collaborating or working with a colleague. Image 342 further emphasizes this sentiment through the facial expressions of the avatar.

The test-taker can provide feedback via graphical element 346 or graphical element 348. For example, graphical element 346 may correspond to a “me” response. In other words, the test-taker relates to the sentiment illustrated in customized image-caption pair 340. Graphical element 348 may correspond to a “not me” response. In other words, the test-taker does not relate to the sentiment illustrated in customized image-caption pair 340.

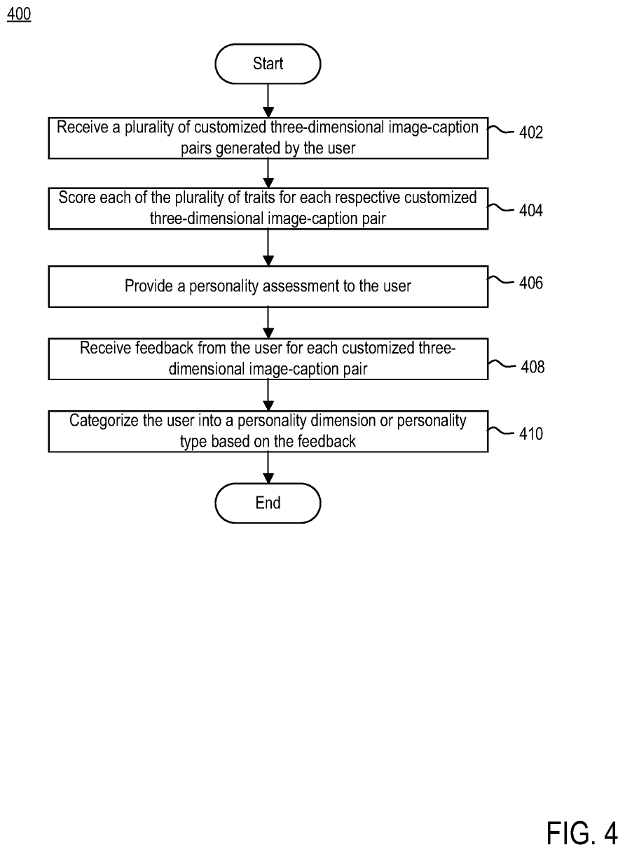

FIG. 4 is a flow diagram illustrating a method 400 of facilitating a personality assessment for a test taker, according to example embodiments. Method 400 may begin at step 402.

At step 402, back-end computing system 104 may receive a plurality of customized three-dimensional image-caption pairs generated by a user or developer. In some embodiments, back-end computing system 104 may receive an upload of a three-dimensional graphic image that was generated by the admin using application 110 executing on admin device 102. In some embodiments, back-end computing system 104 may receive a caption with the three-dimensional graphic image from admin device 102. The caption may describe a trait, sentiment, or idea captured by the image. In some embodiments, each customized three-dimensional image-caption pair is assigned a plurality of traits to each of the customized three-dimensional image-caption pairs. For example, via designer module 206, an admin may provide the various traits that are assigned or tagged to the image-caption pair. Each trait corresponds to an overarching personality dimension or personality type. In some embodiments, an admin or clinician may assign 5-8 personality traits to the image-caption pair. In some embodiments, an admin or clinician may assign more than 8 personality traits the image-caption pair. In some embodiments, an admin or clinician may assign less than 5 personality traits to the image-caption pair.

At step 404, back-end computing system 104 may score each of the plurality of traits for each respective customized three-dimensional image-caption pair. For example, via designer module 206, an admin may score each of the assigned personality traits. For example, an admin may rank each personality trait based on an analysis of the image. The personality trait's rank may correspond to a score associated with that personality trait for that image.

At step 406, back-end computing system 104 may provide a personality assessment to a test taker. In some embodiments, the personality assessment may include a plurality of custom image-caption pairs. For example, personality assessment may include a plurality of image-caption pairs 214 that includes a custom three-dimensional graphic image generated by a user of admin device 102. In some embodiments, personality assessment module 208 may deploy personality assessment via API module 210. For example, if test-taker device 108 accesses personality assessment via a website associated with third party system 106, personality assessment 212 may be delivered to test-taker device 108 through the website via one or more APIs managed by API module 210.

At step 408, back-end computing system 104 may receive feedback from the test taker for each customized three-dimensional image-caption pair in the personality assessment. For example, when a test-taker is presented with a custom image-caption pair, the test-taker may be prompted for feedback. In some embodiments, the test-taker prompt may be “me” or “not me.” In other words, the test-taker may be prompted for feedback regarding whether the custom image-caption pair 214 is the test-taker (“me”) or not the test-taker (“not me”). Back-end computing system 104 may receive the “me” or “not me” feedback via API module 210.

At step 410, back-end computing system 104 may categorize the test-taker into a personality dimension or personality type based on the feedback. Based on the personality trait data, personality assessment module 208 may categorize the test-taker into a personality dimension or personality type. For example, personality assessment module 208 may apply an algorithm to the test-takers feedback, which may aggregate the personality traits and respective scores for each custom image-caption pair 214 and output a personality dimension or personality type.

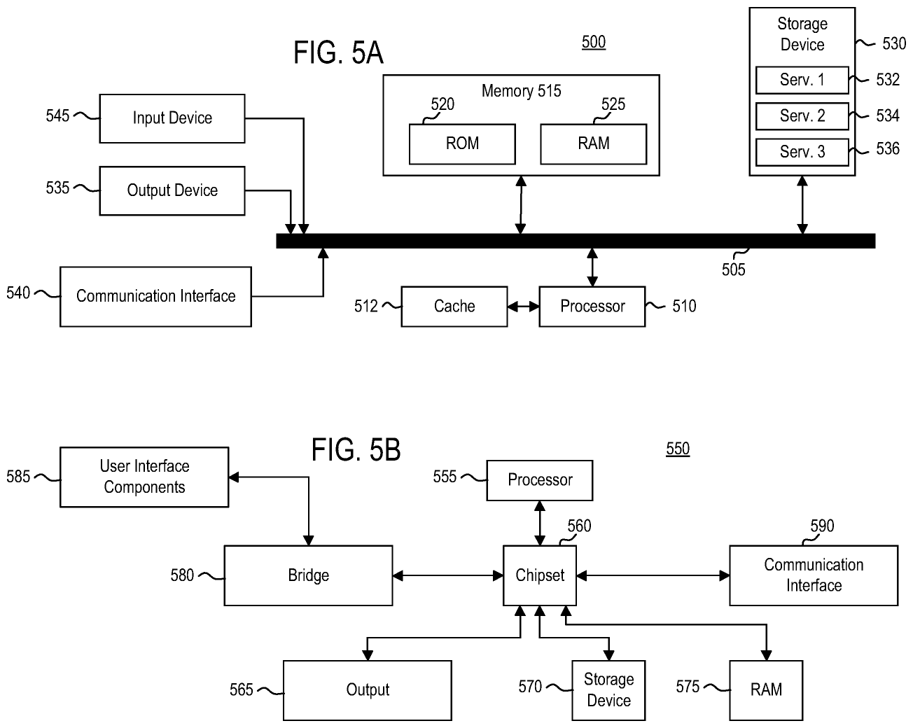

FIG. 5A illustrates an architecture of system bus computing system 500, according to example embodiments. One or more components of system 500 may be in electrical communication with each other using a bus 505. System 500 may include a processor (e.g., one or more CPUs, GPUs or other types of processors) 510 and a system bus 505 that couples various system components including the system memory 515, such as read only memory (ROM) 520 and random-access memory (RAM) 525, to processor 510. System 500 can include a cache of high-speed memory connected directly with, in close proximity to, or integrated as part of processor 510. System 500 can copy data from memory 515 and/or storage device 530 to cache 512 for quick access by processor 510. In this way, cache 512 may provide a performance boost that avoids processor 510 delays while waiting for data. These and other modules can control or be configured to control processor 510 to perform various actions. Other system memory 515 may be available for use as well. Memory 515 may include multiple different types of memory with different performance characteristics. Processor 510 may be representative of a single processor or multiple processors. Processor 510 can include one or more of a general-purpose processor or a hardware module or software module, such as service 1 532, service 2 534, and service 5 536 stored in storage device 530, configured to control processor 510, as well as a special-purpose processor where software instructions are incorporated into the actual processor design. Processor 510 may essentially be a completely self-contained computing system, containing multiple cores or processors, a bus, memory controller, cache, etc. A multi-core processor may be symmetric or asymmetric.

To enable user interaction with the system 500, an input device 545 can be any number of input mechanisms, such as a microphone for speech, a touch-sensitive screen for gesture or graphical input, keyboard, mouse, motion input, speech and so forth. An output device 535 (e.g., a display) can also be one or more of a number of output mechanisms known to those of skill in the art. In some instances, multimodal systems can enable a user to provide multiple types of input to communicate with system 500. Communication interface 540 can generally govern and manage the user input and system output. There is no restriction on operating on any particular hardware arrangement and therefore the basic features here may easily be substituted for improved hardware or firmware arrangements as they are developed.

Storage device 530 may be a non-volatile memory and can be a hard disk or other type of computer readable media that can store data that are accessible by a computer, such as magnetic cassettes, flash memory cards, solid state memory devices, digital versatile disks, cartridges, random access memories (RAMs) 525, read only memory (ROM) 520, and hybrids thereof.

Storage device 530 can include services 532, 534, and 536 for controlling the processor 510. Other hardware or software modules are contemplated. Storage device 530 can be connected to system bus 505. In one aspect, a hardware module that performs a particular function can include the software component stored in a computer-readable medium in connection with the necessary hardware components, such as processor 510, bus 505, output device 535 (e.g., a display), and so forth, to carry out the function.

FIG. 5B illustrates a computer system 550 having a chipset architecture, according to example embodiments. Computer system 550 may be an example of computer hardware, software, and firmware that can be used to implement the disclosed technology. System 550 can include one or more processors 555, representative of any number of physically and/or logically distinct resources capable of executing software, firmware, and hardware configured to perform identified computations. One or more processors 555 can communicate with a chipset 560 that can control input to and output from one or more processors 555. In this example, chipset 560 outputs information to output 565, such as a display, and can read and write information to storage device 570, which can include magnetic media, and solid-state media, for example. Chipset 560 can also read data from and write data to storage device 575 (e.g., RAM). A bridge 580 for interfacing with a variety of user interface components 585 can be provided for interfacing with chipset 560. Such user interface components 585 can include a keyboard, a microphone, touch detection and processing circuitry, a pointing device, such as a mouse, and so on. In general, inputs to system 550 can come from any of a variety of sources, machine generated and/or human generated.

Chipset 560 can also interface with one or more communication interfaces 590 that can have different physical interfaces. Such communication interfaces can include interfaces for wired and wireless local area networks, for broadband wireless networks, as well as personal area networks. Some applications of the methods for generating, displaying, and using the GUI disclosed herein can include receiving ordered datasets over the physical interface or be generated by the machine itself by one or more processors 555 analyzing data stored in storage device 570 or 575. Further, the machine can receive inputs from a user through user interface components 585 and execute appropriate functions, such as browsing functions by interpreting these inputs using one or more processors 555.

It can be appreciated that example systems 500 and 550 can have more than one processor 510 or be part of a group or cluster of computing devices networked together to provide greater processing capability.

While the foregoing is directed to embodiments described herein, other and further embodiments may be devised without departing from the basic scope thereof. For example, aspects of the present disclosure may be implemented in hardware or software or a combination of hardware and software. One embodiment described herein may be implemented as a program product for use with a computer system. The program(s) of the program product define functions of the embodiments (including the methods described herein) and can be contained on a variety of computer-readable storage media. Illustrative computer-readable storage media include, but are not limited to: (i) non-writable storage media (e.g., read-only memory (ROM) devices within a computer, such as CD-ROM disks readably by a CD-ROM drive, flash memory, ROM chips, or any type of solid-state non-volatile memory) on which information is permanently stored; and (ii) writable storage media (e.g., floppy disks within a diskette drive or hard-disk drive or any type of solid state random-access memory) on which alterable information is stored. Such computer-readable storage media, when carrying computer-readable instructions that direct the functions of the disclosed embodiments, are embodiments of the present disclosure.

It will be appreciated to those skilled in the art that the preceding examples are exemplary and not limiting. It is intended that all permutations, enhancements, equivalents, and improvements thereto are apparent to those skilled in the art upon a reading of the specification and a study of the drawings are included within the true spirit and scope of the present disclosure. It is therefore intended that the following appended claims include all such modifications, permutations, and equivalents as fall within the true spirit and scope of these teachings.

1. A method, comprising:
receiving a plurality of customized three-dimensional image-caption pairs generated by an administrator, wherein each three-dimensional image-caption pair is assigned a plurality of traits to each of the plurality of customized three-dimensional image-caption pair;
for each customized three-dimensional image-caption pair, scoring, by a computing system, each trait of the plurality of traits;
deploying, by the computing system, a personality assessment comprising the plurality of customized three-dimensional image-caption pairs;
for each customized three-dimensional image-caption pair, receiving, by the computing system, response data from a test-taker, the response data comprising an indication of whether the test-taker relates to the customized three-dimensional image-caption pair; and
based on the response data, categorizing, by the computing system, the test-taker into a personality dimension or personality type.

2. The method of claim 1, wherein a caption in an image-caption pair describes at least one of a trait, sentiment, or idea illustrated by an image in the image-caption pair.

3. The method of claim 1, wherein scoring, by the computing system, each trait of the plurality of traits comprises:
ranking each trait assigned to the customized three-dimensional image-caption pair based on an analysis of an image.

4. The method of claim 1, wherein deploying, by the computing system, the personality assessment comprising the plurality of customized three-dimensional image-caption pairs comprises:
delivering the personality assessment to the test-taker through a third-party website via one or more application programming interfaces associated with the computing system.

5. The method of claim 1, wherein the response data comprises a binary response to each customized three-dimensional image-caption pair.

6. The method of claim 1, wherein categorizing, by the computing system, the test-taker into the personality dimension or the personality type comprises:
aggregating the traits and respective scores for the traits for each customized three-dimensional image-caption pair.

7. The method of claim 1, further comprising:
prior to deploying the personality assessment, posting, by the computing system, the plurality of customized three-dimensional image-caption pairs for testing to determine whether each customized three-dimensional image-caption pair accurately conveys an intent of the customized three-dimensional image-caption pair.

8. A non-transitory computer readable medium comprising one or more sequences of instructions, which, when executed by one or more processors, causes a computing system to perform operations comprising:
receiving, by the computing system, a plurality of customized three-dimensional image-caption pairs generated by an administrator, wherein each customized three-dimensional image-caption pair is assigned a plurality of traits to each of the plurality of customized three-dimensional image-caption pairs;
for each customized three-dimensional image-caption pair, scoring, by the computing system, each trait of the plurality of traits;
deploying, by the computing system, a personality assessment comprising the plurality of customized three-dimensional image-caption pairs;
for each customized three-dimensional image-caption pair, receiving, by the computing system, response data from a test-taker, the response data comprising an indication of whether the test-taker relates to the customized three-dimensional image-caption pair; and
based on the response data, categorizing, by the computing system, the test-taker into a personality dimension or personality type.

9. The non-transitory computer readable medium of claim 8, wherein a caption in an image-caption pair describes at least one of a trait, sentiment, or idea illustrated by an image in the image-caption pair.

10. The non-transitory computer readable medium of claim 8, wherein scoring, by the computing system, each trait of the plurality of traits comprises:
ranking each trait assigned to the customized three-dimensional image-caption pair based on an analysis of an image.

11. The non-transitory computer readable medium of claim 8, wherein deploying, by the computing system, the personality assessment comprising the plurality of customized three-dimensional image-caption pairs comprises:
delivering the personality assessment to the test-taker through a third-party website via one or more application programming interfaces associated with the computing system.

12. The non-transitory computer readable medium of claim 8, wherein the response data comprises a binary response to each customized three-dimensional image-caption pair.

13. The non-transitory computer readable medium of claim 8, wherein categorizing, by the computing system, the test-taker into the personality dimension or the personality type comprises:
aggregating the traits and respective scores for the traits for each customized three-dimensional image-caption pair.

14. The non-transitory computer readable medium of claim 8, further comprising:
prior to deploying the personality assessment, posting, by the computing system, the plurality of customized three-dimensional image-caption pairs for testing to determine whether each customized three-dimensional image-caption pair accurately conveys an intent of the customized three-dimensional image-caption pair.

15. A system comprising:
a processor; and
a memory having programming instructions stored thereon, which, when executed by the processor, causes the system to perform operations comprising:
receiving a plurality of customized three-dimensional image-caption pairs generated by an administrator, wherein each customized three-dimensional image-caption pair is assigned a plurality of traits to each of the plurality of customized three-dimensional image-caption pairs;
for each customized three-dimensional image-caption pair, scoring each trait of the plurality of traits;
deploying a personality assessment comprising the plurality of customized three-dimensional image-caption pairs;
for each customized three-dimensional image-caption pair, receiving response data from a test-taker, the response data comprising an indication of whether the test-taker relates to the customized three-dimensional image-caption pair; and
based on the response data, categorizing the test-taker into a personality dimension or personality type.

16. The system of claim 15, wherein a caption in an image-caption pair describes at least one of a trait, sentiment, or idea illustrated by an image in the image-caption pair.

17. The system of claim 15, wherein scoring each trait of the plurality of traits comprises:
ranking each trait assigned to the customized three-dimensional image-caption pair based on an analysis of an image.

18. The system of claim 15, wherein deploying the personality assessment comprising the plurality of customized three-dimensional image-caption pairs comprises:
delivering the personality assessment to the test-taker through a third-party website via one or more application programming interfaces associated with the system.

19. The system of claim 15, wherein categorizing the test-taker into the personality dimension or the personality type comprises:
aggregating the traits and respective scores for the traits for each customized three-dimensional image-caption pair.

20. The system of claim 15, wherein the operations further comprise:
prior to deploying the personality assessment, posting the plurality of customized three-dimensional image-caption pairs for testing to determine whether each customized three-dimensional image-caption pair accurately conveys an intent of the customized three-dimensional image-caption pair.
